(19) United States
(12) Patent Application Publication
(10) Pub. No.: US 2018/0369519 A1
(43) Pub. Date: Dec. 27, 2018

(54) Inhalation Device With Multi Chamber Container

(71) Applicant: Resolve Digital Health Inc., Toronto (CA)

(72) Inventor: Robert ADELSON, Vaughn (CA)

(73) Assignee: Resolve Digital Health Inc., Toronto (CA)

(21) Appl. No.: 16/019,029

(22) Filed: Jun. 26, 2018

Related U.S. Application Data
(60) Provisional application No. 62/525,572, filed on Jun. 27, 2017.

(51) Int. Cl. A61M 15/00; G05B 15/02

(52) U.S. Cl. CPC A61M 15/0066; G05B 15/02; A61M 15/0086; A61M 15/0003; A61M 2205/50; A61M 11/042; A61M 2205/3584; A61M 2205/505; A61M 2205/103; A61M 2205/6009; A61M 2205/273

(57) ABSTRACT

The present disclosure relates to an inhaler having a container, wherein the container can include a plurality of chambers, each chamber of the plurality of chambers being filled with a liquid in a manner such that upon actuation of said inhaler, and based on selection of dosing parameter by a user, one or more liquids from respective chambers are retrieved and dispensed to said user through an opening.

### TECHNICAL FIELD

The present disclosure relates generally to the field of inhalers for herbal oils/cannabis extracts for medication/recreational purposes. In particular, the present disclosure relates to a device that can store multiple herbal oils/plant extracts, and selectively dispense them.

### BACKGROUND

Background description includes information that may be useful in understanding the present invention. It is not an admission that any of the information provided herein is prior art or relevant to the presently claimed invention, or that any publication specifically or implicitly referenced is prior art.

In view of high cost and side effects of allopathic medicines, many people prefer to depend on alternate/traditional medicines at least for minor ailments such as nausea, colds and flues, cough, headache, aches and pains, gastroenterology disorders (such as constipation and diarrhea), menstrual cramps, insomnia skin disorders and dandruff. Also many physicians believe in usefulness of alternative medicines especially in the cases where conventional medicine cannot provide a cure. Their faith in alternate medicines, especially in herbal medicines, is strengthened by the fact that in modern medicine many drugs in common use today have botanical origins.

Herbs are available in many forms depending on their medicinal purpose and the body system involved. Herbs may be prepared as tinctures or extracts, capsules or tablets, lozenges, teas, juices, oils and waxes for vapor and inhalation treatment or bath products. Inhaled phytotherapies have been in use for treatment of respiratory diseases such as asthma, respiratory infections, sinus conditions and tracheitis. The treatment helps to open congested sinuses and lung passages, promote mucus discharge and ease breathing. Delivering herbal medicines directly to the target sites in airways offers high local drug concentration without first pass effect, rapid onset of drug action, minimum systemic effects and reduced side effects.

Besides using for respiratory ailments, pulmonary herbal medicine delivery by inhalation may also be employed for systemic drugs because large surface area, extensive vascularization and thin alveolar epithelium of lungs facilitate direct drug absorption in blood circulation. For this reason inhalation phytotherapies by means of smoke has been known to have existed in India way back in 2000-3000 BC when inhalation of smoke of Atropa Belladonna leaves was practiced for medicinal purposes. Inhalation phytotherapies by means of smoke, steam or vapor are still prevalent in Africa, India, China and many other Asian countries.

Modern research in medicines and consequent formulation designs has provided many inhalable pharmaceutical products along with efficient delivery means such as inhalers as means for delivering medication into the body via the lungs. The most common is the pressurized metered-dose inhaler (MDI). In MDIs, medication is typically stored in solution in a pressurized canister that contains a propellant, although it may also be a suspension. The MDI canister is attached to a hand-operated actuator. On activation, the MDI releases a fixed dose of medication in aerosol form. However, conventional inhalers such as the MDIs are not suited for delivering medication that is present in plants and herbs. This is particularly the case where patients have been prescribed cannabis for treating various indications such as glaucoma, nausea, and other symptoms.

Herbal inhalable medicines are typically available as oils and waxes and current technology requires one container for one type of medicine. Patients are required to purchase and transport these medicines in separate containers from which doses can be consumed using inhalators that are available for the purpose. When required to inhale a herbal medicine a user has to insert the corresponding container in a inhalation device and take a dose of the medicine. If a user needs to inhale a combination of herbal medicines, he must remove one container and replace it with another container in order to ingest another medicine. Current technology does not allow for the consistent, metered-dosing of the oil/wax of a combination of herbal medicines. Thus the traditional devices and method is not convenient from users' point of view because firstly he has to carry a number of containers for these medicines and secondly ingestion is a cumbersome process wherein he has to remember combination of different oils/waxes and take them one by one.

There is, therefore, need in the art for an improved container for inhalable herbal medicines and an improved inhaler that can mitigate the drawbacks of the conventional method and devices and allows patients to use only one device to store and dispense multiple oils/waxes and dispense them for inhalation.

### SUMMARY OF THE INVENTION

Aspects of the present disclosure relate to storage and dispensing of herbal oils and waxes for inhalation therapy. While inhalers that deliver doses of herbal oils and waxes for inhalation are well known in art, they are designed to store and dispense only one herbal medicine at a time requiring patient to carry different oils in multiplicity of containers. Patients are also required to change the container in the inhaler device if he is required to take doses of multiple medicines in combination or at different times. The present disclosure provides an inhaler that incorporates a container capable of storing more than one type of oils and the inhaler can deliver any of these medicines either singly or in combination for inhalation depending symptoms and requirement of treatment.

In an aspect, the container of the disclosed inhalation device can be made of glass, plastic or metal and can incorporate more than one chamber. Each of the chambers can be filled with an herbal medicine in liquid or glass form thus enabling storage of multiple medicines that a patient may be required to ingest either individually or in a combination, depending on symptoms. The patient can connect the container to the inhalation device and then select/the medicine(s) that is/are required, alone or in combination for inhalation.

The medicines stored in chambers of the container can last for number of doses sufficient for few days thus enabling a user to carry his medicine requirement in a convenient manner especially during occasions such as travelling when carrying number of containers and connecting them to the inhalation device for ingestion can be troublesome and inconvenient.

In an aspect, the disclosed inhalation device incorporates a rotary device that can rotate the container and position the desired chamber of the container for dispensing the selected medicine for inhalation depending on selection made by the user. In another aspect, the rotary device can allow for individual dose or a combination of doses.

In an aspect, the disclosed inhalation device can incorporate wireless communication means such as Bluetooth to get connected to a computing device such as a smart phone, a laptop or a desktop to name a few. An application in the communication device can enable the size of the dose of each of the medicines to be metered and varied depending on requirement. Thus the doses can be customized depending on what is appropriate for the patient.

In an aspect, the proposed disclosure pertains to an inhaler comprising a container, said container comprising a plurality of chambers, each chamber of said plurality of chambers being filled with a liquid in a manner such that upon actuation of said inhaler, and based on selection of dosing parameter by a user, one or more liquids from respective chambers are retrieved and dispensed to said user through an opening.

In another aspect, the one or more retrieved liquids can at least be partially admixed before being dispensed to said user.

In yet another aspect, the one or more retrieved liquids can be dispensed separately to said user through the opening, wherein each of said plurality of chambers are directly connected to said opening at their one end.

In an aspect, liquid in a first chamber of said plurality of chambers can be a medicament. In another aspect, the liquid in a second chamber of the plurality of chambers can be an antidote of said medicament. The proposed device can further include a communication module that can be operatively coupled with a portable computing device (such as a mobile phone, smart phone, tablet pc, wearable device, or a laptop), and wherein upon receipt of a second signal from said portable computing device, said antidote can be mixed with said medicament. The proposed inhaler can further include a communication module that can be operatively coupled with a portable computing device, wherein upon receipt of a third signal from the portable computing device, the first chamber containing the medicament can be ruptured or rendered inaccessible to the user, wherein the third signal can be transmitted from the portable computing device when operation of said inhaler is not intended.

In an aspect, the liquid can be in gaseous form. In another aspect, the liquid can be any or a combination of a herbal oil or a wax. In yet another aspect, the liquid can be cannabis oil or cannabis extract.

In another aspect, the inhaler can include a communication module that can be operatively coupled with a portable computing device, wherein the inhaler can be actuated upon receipt of a signal from the portable computing device. In another aspect, the inhaler can be configured in a manner such that the one or more retrieved liquids from the respective chambers are mixed in a configurable proportion, said configuration of liquid proportion being done through any or a combination of an interface provided on the inhaler, or through one or more signals sent by a portable computing device that said inhaler is operatively coupled to.

In an aspect, the container can be made of any or a combination of a glass, plastic, or a metal.

in another aspect, the dosing parameter can be selected by the user through any or a combination of an interface provided on the inhaler, or through one or more signals sent by a portable computing device that said inhaler is operatively coupled to, wherein said one or more signals are transmitted based on selection of an option through an application that is configured in the portable computing device. The dosing parameter can be pre-configured and automatically selected upon actuation of the inhaler. Furthermore, the dosing parameter can be indicative of any or a combination of liquids that need to be combined during dispensation through the opening, whether said liquids are to be dispensed as a combination or separately, and proportion/strength of each liquid to be dispensed.

In an aspect, the proposed inhaler can include a rotary device that can be configured to rotate the container, and position the one or more chambers of the container for dispensing the selected liquids based on the received dosing parameter.

The proposed disclosure further relates to a method to dispense one or more liquids through an opening of an inhaler, said method comprising the steps of: receiving, at the inhaler, an actuation signal to activate said inhaler, said inhaler comprising a container that further comprises a plurality of chambers, each chamber of said plurality of chambers being filled with a liquid; and receiving, at the inhaler, selection of a dosing parameter based on which one or more liquids from respective chambers are retrieved and dispensed to said user through an opening.

Various objects, features, aspects and advantages of the inventive subject matter will become more apparent from the following detailed description of preferred embodiments, along with the accompanying drawing figures in which like numerals represent like components.

### BRIEF DESCRIPTION OF THE DRAWINGS

The accompanying drawings are included to provide a further understanding of the present disclosure, and are incorporated in and constitute a part of this specification. The drawings illustrate exemplary embodiments of the present disclosure and, together with the description, serve to explain the principles of the present disclosure.

FIG. 1 illustrates an exemplary block diagram indicating various functional parts/modules of an inhalation device with multi chamber oil container in accordance with embodiments of the present disclosure.

FIGS. 2A to 2C illustrate exemplary representations of the proposed multi-chamber container in accordance with embodiments of the present disclosure.

FIG. 3 illustrates another exemplary representation of the proposed multi-chamber container in accordance with embodiments of the present disclosure.

FIG. 4 illustrates an exemplary representation of the proposed inhaler in accordance with embodiments of the present disclosure.

### DETAILED DESCRIPTION

The following is a detailed description of embodiments of the disclosure depicted in the accompanying drawings. The embodiments are in such detail as to clearly communicate the disclosure. However, the amount of detail offered is not intended to limit the anticipated variations of embodiments; on the contrary, the intention is to cover all modifications, equivalents, and alternatives falling within the spirit and scope of the present disclosure as defined by the appended claims.

Unless the context requires otherwise, throughout the specification which follow, the word “comprise” and variations thereof, such as, “comprises” and “comprising” are to be construed in an open, inclusive sense that is as “including, but not limited to.”

Reference throughout this specification to “one embodiment” or “an embodiment” means that a particular feature, structure or characteristic described in connection with the embodiment is included in at least one embodiment. Thus, the appearances of the phrases “in one embodiment” or “in an embodiment” in various places throughout this specification are not necessarily all referring to the same embodiment. Furthermore, the particular features, structures, or characteristics may be combined in any suitable manner in one or more embodiments.

As used in this specification and the appended claims, the singular forms “a,” “an,” and “the” include plural referents unless the content clearly dictates otherwise. It should also be noted that the term “or” is generally employed in its sense including “and/or” unless the content clearly dictates otherwise.

In some embodiments, the numbers expressing quantities of ingredients, properties such as concentration, reaction conditions, and so forth, used to describe and claim certain embodiments of the invention are to be understood as being modified in some instances by the term “about.” Accordingly, in some embodiments, the numerical parameters set forth in the written description are approximations that can vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the invention are approximations, the numerical values set forth in the specific examples are reported as precisely as practicable.

The recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g. “such as”) provided with respect to certain embodiments herein is intended merely to better illuminate the invention and does not pose a limitation on the scope of the invention otherwise claimed. No language in the specification should be construed as indicating any non-claimed element essential to the practice of the invention.

The headings and abstract of the invention provided herein are for convenience only and do not interpret the scope or meaning of the embodiments.

Various terms as used herein. To the extent a term used in a claim is not defined below, it should be given the broadest definition persons in the pertinent art have given that term as reflected in printed publications and issued patents at the time of filing.

The present disclosure relates generally to storage and dispensing of herbal oils and waxes for inhalation therapy. Conventional inhalers are designed to store and dispense only one herbal medicine at a time requiring patient to carry different oils in multiplicity of containers. Patients are also required to change the container in the inhaler device if he is required to take doses of multiple medicines in combination or at different times. The present disclosure provides an inhaler that incorporates a multi chamber container capable of storing more than one type of oils and the inhaler can deliver any of these medicines either singly or in combination for inhalation depending symptoms and requirement of treatment.

In an embodiment, each of the chambers of the multi chamber container of the disclosed inhalation device can be filled with an herbal medicine in liquid or glass form thus enabling storage of multiple medicines that a patient may be required to ingest either individually or in a combination, depending on symptoms. The patient can connect the container to the inhalation device and then select/the medicine(s) that is/are required, alone or in combination for inhalation.

In an embodiment, the disclosed inhalation device incorporates a rotary device that can rotate the container and position the desired chamber of the container for dispensing a metered dose of the selected medicine for inhalation depending on selection made by the user. In another embodiment, the rotary device can allow for individual dose or a combination of doses wherein when a combination of medicines are to be ingested by the patient, the device can enable desired chambers that store the required medicines to be positioned sequentially for dispensing metered doses of each of them thus enabling the patient to ingest combination of medicines.

In an aspect, the proposed disclosure pertains to an inhaler comprising a container, said container comprising a plurality of chambers, each chamber of said plurality of chambers being filled with a liquid in a manner such that upon actuation of said inhaler, and based on selection of dosing parameter by a user, one or more liquids from respective chambers are retrieved and dispensed to said user through an opening.

In another aspect, the one or more retrieved liquids can at least be partially admixed before being dispensed to said user.

In yet another aspect, the one or more retrieved liquids can be dispensed separately to said user through the opening, wherein each of said plurality of chambers are directly connected to said opening at their one end.

In an aspect, liquid in a first chamber of said plurality of chambers can be a medicament. In another aspect, the liquid in a second chamber of the plurality of chambers can be an antidote of said medicament. The proposed device can further include a communication module that can be operatively coupled with a portable computing device (such as a mobile phone, smart phone, tablet pc, wearable device, or a laptop), and wherein upon receipt of a second signal from said portable computing device, said antidote can be mixed with said medicament. The proposed inhaler can further include a communication module that can be operatively coupled with a portable computing device, wherein upon receipt of a third signal from the portable computing device, the first chamber containing the medicament can be ruptured or rendered inaccessible to the user, wherein the third signal can be transmitted from the portable computing device when operation of said inhaler is not intended.

In an aspect, the liquid can be in gaseous form. In another aspect, the liquid can be any or a combination of a herbal oil or a wax. In yet another aspect, the liquid can be cannabis oil or cannabis extract.

In another aspect, the inhaler can include a communication module that can be operatively coupled with a portable computing device, wherein the inhaler can be actuated upon receipt of a signal from the portable computing device. In another aspect, the inhaler can be configured in a manner such that the one or more retrieved liquids from the respective chambers are mixed in a configurable proportion, said configuration of liquid proportion being done through any or a combination of an interface provided on the inhaler, or through one or more signals sent by a portable computing device that said inhaler is operatively coupled to.

In an aspect, the container can be made of any or a combination of a glass, plastic, or a metal.

in another aspect, the dosing parameter can be selected by the user through any or a combination of an interface provided on the inhaler, or through one or more signals sent by a portable computing device that said inhaler is operatively coupled to, wherein said one or more signals are transmitted based on selection of an option through an application that is configured in the portable computing device. The dosing parameter can be pre-configured and automatically selected upon actuation of the inhaler. Furthermore, the dosing parameter can be indicative of any or a combination of liquids that need to be combined during dispensation through the opening, whether said liquids are to be dispensed as a combination or separately, and proportion/strength of each liquid to be dispensed.

In an aspect, the proposed inhaler can include a rotary device that can be configured to rotate the container, and position the one or more chambers of the container for dispensing the selected liquids based on the received dosing parameter.

The proposed disclosure further relates to a method to dispense one or more liquids through an opening of an inhaler, said method comprising the steps of: receiving, at the inhaler, an actuation signal to activate said inhaler, said inhaler comprising a container that further comprises a plurality of chambers, each chamber of said plurality of chambers being filled with a liquid; and receiving, at the inhaler, selection of a dosing parameter based on which one or more liquids from respective chambers are retrieved and dispensed to said user through an opening.

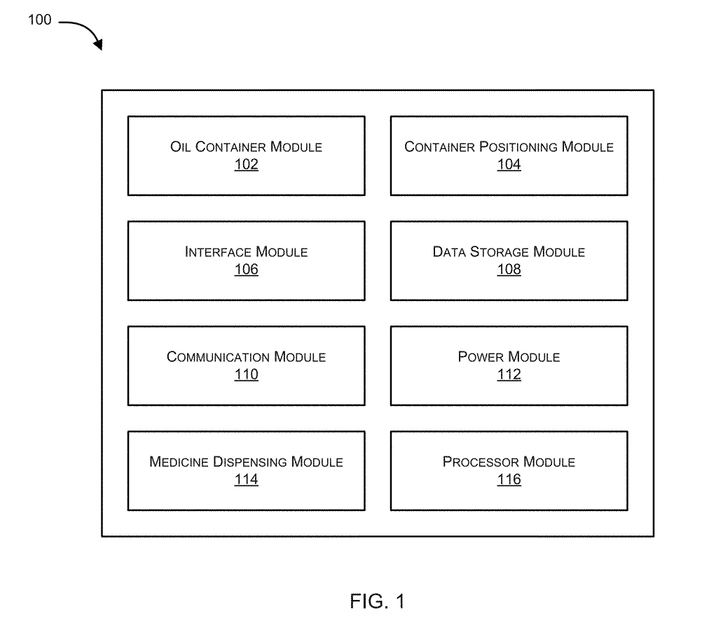

Referring now to FIG. 1 that illustrates an exemplary block diagram 100 indicating various functional subsystems of the disclosed inhaler device with multi-chamber container. The device of the present disclosure can incorporate an oil container module 102, a container positioning module 104, an interface module 106, a data storage module 108, a communication module 110, a power module 112, a medicine dispensing module 114 and a processer module 116.

In an embodiment, the oil container module 102 can be a multi chamber container that can store one or more one herbal medicines. It is to be appreciated that reference to the term herbal medicine in the instant disclosure is to be interpreted to include any plant extract including but not limited to cannabis oil/extract.

In an aspect, the proposed container can be made of glass, plastic or metal and can incorporate more than one chamber. Each of the chambers can be filled with a herbal medicine in liquid or gaseous form, enabling storage of multiple medications/liquids/gases that a patient or a user (for recreational purposes) may be required to ingest either individually or in a combination, depending on symptoms. The medicines stored in chambers of the container can last for number of doses sufficient for few days thus enabling a user to carry his medicine requirement in a convenient manner especially during occasions such as travelling when carrying number of containers and connecting them to the inhalation device for ingestion can be troublesome and inconvenient. The patient can connect the container to the inhalation device and then select/the medicine(s) that is/are required, alone or in combination for inhalation. In an embodiment, the container can remain connected to the device thus a user is required to carry only one item to meet his medicine needs.

In an embodiment, the different chambers in the container can have separate identity and can be identified by assigning unique ID such as a number and details regarding medicines stored in each of these chambers can be stored by user in the device. Alternatively different chambers can be assigned for different medicines beforehand and mentioned on the container against each of the chambers so that the user fills up different medicines in respective assigned chambers.

In an aspect, the proposed inhaler device and/or the container that forms part of said inhaler can be operatively coupled with a portable computing device such as mobile/smart phone or a wearable device, and enable configuration of the operation of the each of the chambers that form part of the container. For instance, the portable computing device can issue signals that can configure construction of the chambers to change so as to lock or unlock one or more chambers, or even open one or more valves that form part of the chambers so as to allow admixture of one or more liquids that form part of the chambers.

In an embodiment, the proposed container can be configured to fit into the device only in one position so that orientation of each of the chambers in relation with a reference position is known so as to enable positioning of any particular chamber in a desired position to dispense the medicine stored in that chamber.

In a preferred embodiment, different chambers of the container can be configured in a circular position around a circle so that rotation of the container can enable positioning a chosen chamber in a preferred position, wherein the preferred position (referred to as dispensing position hereinafter) is one in which medicine stored in that chamber can be dispensed.

In an embodiment, the container positioning module 104 can be configured to position the container such that a chamber storing a desired medicine is in the dispensing position. The container positioning module 104 can be a rotary device configured to hold the container and rotate it to bring the desired chamber in in the dispensing position. The rotary device can incorporate an electrically driven motor such as a stepper motor configured to rotate the container by precise degrees and sensors configured to sense angular position of the container based on which rotational movement can be worked out.

In an embodiment, the interface module 106 can be configured to enable user to interact with the device. The interaction can be to activate the dispenser to dispense a dose, to enter symptoms or physiological parameters of the user before and after a dose, and other like information. In an embodiment, the interface module 106 can incorporate a touch screen for user to view and also enter different data and information that the dispenser may provide or may be required to process or store.

In an embodiment, the data storage module 108 can be configured to store information such as details of medicines stored in different chambers, details of timing and frequency of doses, symptoms and/or physiological parameters before and after a dose etc.

In an embodiment, the communication module 110 can be configured to communicate with a computing device such as a smart phone, a laptop or a desktop to name a few. It can incorporate a wireless communication means such as Bluetooth to get connected to the computing device. In an embodiment, communication with a computing device can enable the disclosed device to access an application in the communication device. The application in the computing device can enable the size of the dose of each of the medicines to be metered and varied depending on requirement. Thus the doses can be customized depending on what is appropriate for the patient.

In an embodiment, the power module 112 can be configured to meet power requirement of other functional modules of the device such as to drive the electrical motor of the container positioning module 104. The power module can be a rechargeable battery with associated charging port and circuitry to enable its charging and supplying power to other functional parts at their voltage ratings.

In an embodiment, the medicine dispensing module 114 can be configured to function after a desired chamber of the container has been positioned in dispensing position. It can dispense a metered dose of the stored medicine for ingestion by user. In an embodiment it can dispense metered doses of different medicines from different chambers of the container and thereafter present them for ingestion.

In an embodiment, the medicine dispensing module 114 can incorporate means to enable vaporization of the medicines dispensed from the chambers for inhalation by the user.

In an embodiment, the processer module 116 can be configured to process information received from the computing device based application and control the container positioning module 104 and the medicine dispensing module 114 to enable delivery of required dose in accordance with recommendation from the application.

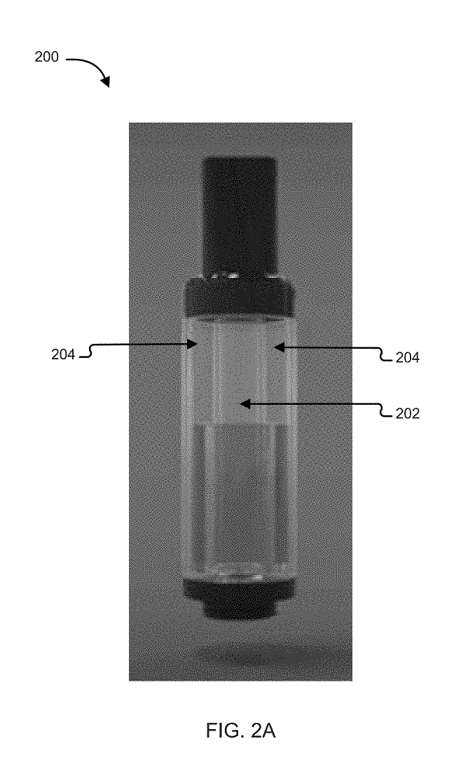

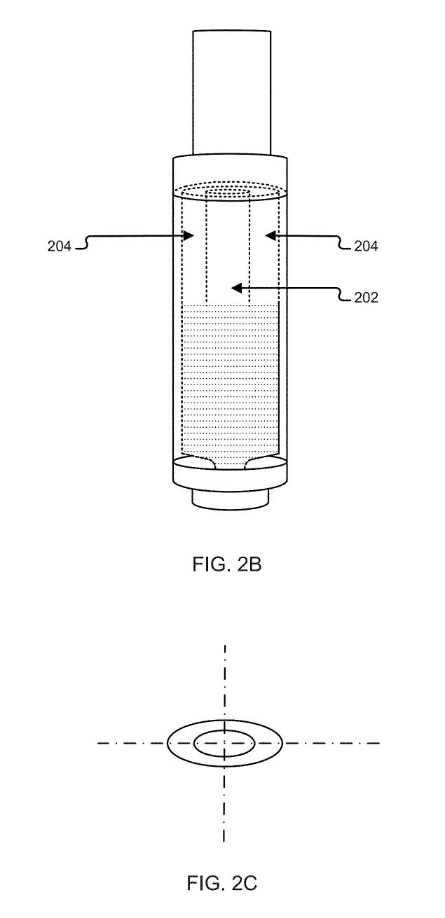

FIGS. 2A to 2C illustrate exemplary representations of the proposed multi-chamber container 200 in accordance with embodiments of the present disclosure. The container 200 can store one or more herbal medicines. In an aspect, the proposed container 200 can be made of glass, plastic, or metal and can incorporate more than one chamber.

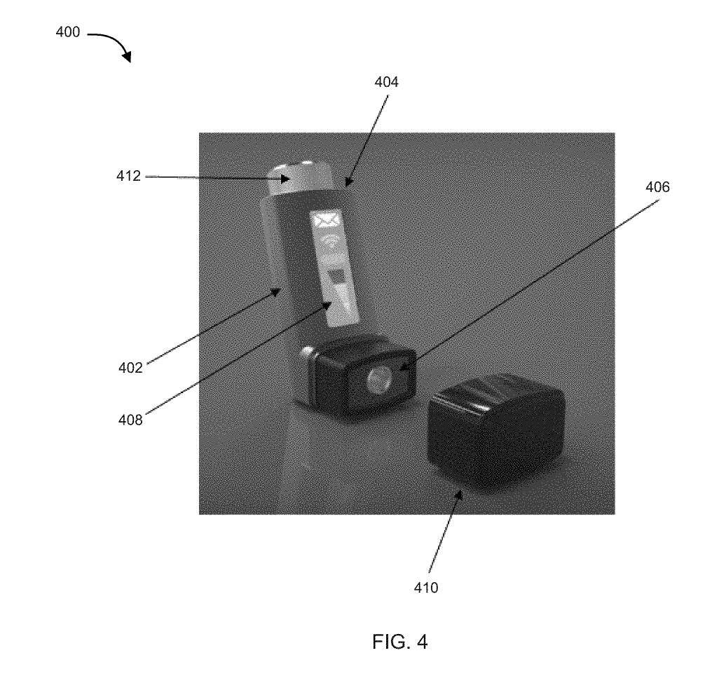

As presented more clearly in FIG. 2B, the proposed container 200 can include two concentric chambers 202 and 204, wherein chamber 202 can store a different liquid/gas/medicament from the one stored in chamber 204. It should be clearly appreciated that although the present disclosure has been represented through concentric chambers 202/204, the container can surely include chambers that are, for instance, parallel to each other or positioned/configured in any other configuration/dimension/shape, all of which possible combinations are well within the scope of the present disclosure. As mentioned above, one or more of the chambers can be controlled remotely through a portable device or through the interface on the inhaler (see FIG. 4) in terms of the amount of liquid being used therefrom for forming the final medicament being delivered to the user, or in terms of being ruptured when the chamber is to be rendered non-operation, or in terms of the size of the vaporized droplet being formed, or in terms of the concentration of the liquid/medicament being used, etc. All such parameters can be controlled so as to generate a desired amount/size/quantity/composition of deliverable/vaporized droplet for the user.

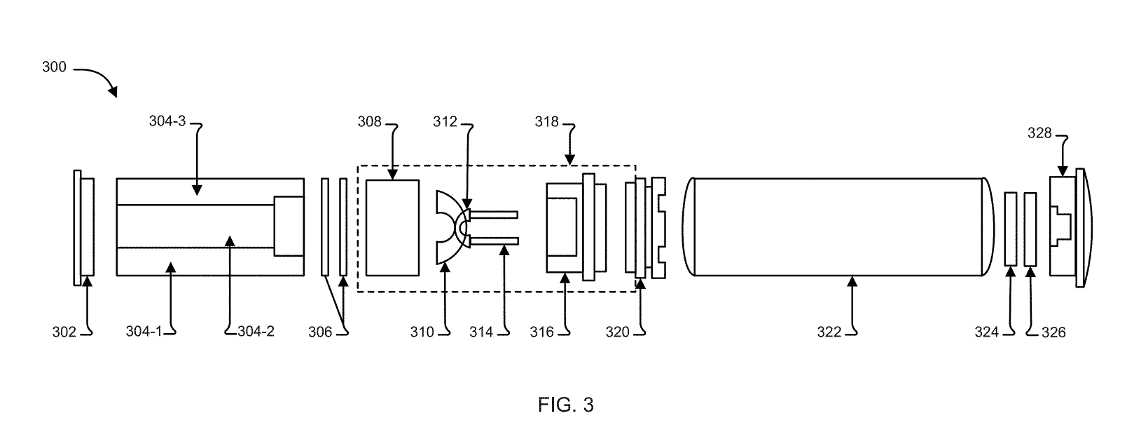

As regards the flexibility of shape/configuration of the chambers within the container, with reference to FIG. 3, the proposed inhaler/dispenser device 300 can include a container 304 that can include 3 cylindrical chambers 304-1, 304-2, and 304-3, each of which has an opening/valve that can be connected to a mouthpiece 302. Each chamber 304-1, 304-2, and 304-3 can be configured to store the same or a different liquid medicament, which can be dispensed to the user through the mouthpiece 302 either after being admixed or can be dispensed separately through the mouthpiece. The proposed inhaler (also interchangeably referred to as vaporization device) 300 can include a vaporization chamber 318 that can include any or a combination of, in a non-limiting manner, one or more filters 306, a heat shield 308, a wick 310, a heating coil 312, wire leads 314, divider 316, and a base 320. The proposed dispensing device 300 can further include a battery 322, an actuator 324, one or more electronic components 324, and an end cap 326. While each of the primary components are shown as separate entities in the figure, they may overlap, be attached, or combined or partially combined. The overall shape of the vaporization unit will generally be cylindrical, though other shapes are possible. One or more of the components may be enclosed or partially enclosed in the housing. The substance container and other components of the inhaler may be sealed to prevent or minimize any leakage of substance. In an exemplary aspect, the container 304 may be generally a cylinder or a shape corresponding to the overall shape of the inhaler. The substance container may also be shaped so as to allow airflow to travel between it and the housing to the mouthpiece. In an alternate embodiment, the substance container is removable from the inhaler. Also, in another additional embodiment, instead of removing the container per se, one or more chambers therein can be rendered to be replaceable so that only the desired chamber can be replaced with the remaining container being in-tact. In another embodiment, each chamber of the container can include a unique identifier using which it can be communicate with the portable computing device for getting configured/controlled. The proposed chamber can further include its own communication tracking mechanism such as Radio-Frequency Identification (RFID) tag, chip or Near Field Communications (NFC) tag or bar code.

Each of the chambers can be filled with an herbal medicine in liquid or glass form thus enabling storage of multiple medicines that a patient may be required to ingest either individually or in a combination, depending on symptoms. The medicines stored in chambers of the container can last for number of doses sufficient for few days thus enabling a user to carry his medicine requirement in a convenient manner especially during occasions such as travelling when carrying number of containers and connecting them to the inhalation device for ingestion can be troublesome and inconvenient. The patient can connect the container 200 to the inhalation device and then select/the medicine(s) that is/are required, alone or in combination for inhalation. In an embodiment, the container 200 can remain connected to the device thus a user is required to carry only one item to meet his medicine needs.

In an embodiment, the different chambers in the container 200 can have separate identity and can be identified by assigning unique ID such as a number and details regarding medicines stored in each of these chambers can be stored by user in the device. Alternatively different chambers can be assigned for different medicines beforehand and mentioned on the container 200 against each of the chambers so that the user fills up different medicines in respective assigned chambers.

In an embodiment, the container 200 can be configured to fit into the device only in one position so that orientation of each of the chambers in relation with a reference position is known so as to enable positioning of any particular chamber in a desired position to dispense the medicine stored in that chamber.

In a preferred embodiment, different chambers of the container 200 can be configured in a circular position around a circle so that rotation of the container 200 can enable positioning a chosen chamber in a dispensing position, wherein the dispensing position is one in which medicine stored in that chamber can be dispensed.

FIG. 4 illustrates an exemplary representation 400 of the proposed inhaler in accordance with embodiments of the present disclosure. As shown, the housing 402 of the inhaler 400 can include a mouthpiece end 406 at one end, and a receiving end 404 at the other end. The receiving end 404 can be adapted to receive a pressurized aerosol container 412 (interchangeably referred to as container in the present specification/disclosure) containing a predetermined number of unit doses of medication. The aerosol container 412 can include a plurality of chambers such that upon actuation of the container by being depressed from the top, metered doses of medication (formed through admixture of liquid container in two or more chambers of the container) in aerosol are discharged form through the mouthpiece 406 for inhalation by a patient The exemplary screen 408 provided on the housing discharge 402 of the inhaler can indicate status of Wi-Fi, GSM connectivity, effectiveness of medication dose and battery status. The screen 408 can be further used to display and/or enter different data and information that the inhaler may provide or may be required to process or store. The cap 410 can be used to cover the mouthpiece 406 to prevent contaminants from entering the interior of the inhaler and to keep the mouthpiece clean.

Thus, the disclosed inhalation device with multi chamber herbal oil container provides a convenient and efficient way to store and administer doses of herbal oils and waxes for inhalation. It further enables customization of doses of herbal inhalable medicines either singly or in combination depending on symptoms through an application in a computing device such as a smart phone or tablet to name a few.

In an aspect, one or more chambers configured in the container of the proposed inhaler can include valves/openings that can be actuated/opened/closed/controlled based on instructions received either through the display interface of the inhaler itself or through a portable computing device such as a smart phone, wherein upon such actuation and the instruction that forms part thereof, the valves can be opened up so as to allow admixture of the liquids. For instance, one chamber can include a medicament such as a cannabis extract, and another chamber includes the antidote thereof, and therefore whenever the user believes that the inhaler has been stolen or can be used in unintended manner, a signal can be sent remotely to the inhaler so as to actuate the valves (or any other appropriate means) and enable mixture of the two liquids from the respective chambers, leaving the cannabis extract such that it would not have any impact on the consuming user. Similarly, any other purpose for which a chamber of the multiple chambers needs to be controlled or locked or rendered non-operational or ruptured, can be achieved either through configuration of the inhaler through the interface of the inhaler or through a wireless/Bluetooth connection between the inhaler and the portable computing device.

While the foregoing describes various embodiments of the invention, other and further embodiments of the invention may be devised without departing from the basic scope thereof. The scope of the invention is determined by the claims that follow. The invention is not limited to the described embodiments, versions or examples, which are included to enable a person having ordinary skill in the art to make and use the invention when combined with information and knowledge available to the person having ordinary skill in the art.

We claim:

1. An inhaler comprising a container, said container comprising a plurality of chambers, each chamber of said plurality of chambers being filled with a liquid in a manner such that upon actuation of said inhaler, and based on selection of dosing parameter by a user, one or more liquids from respective chambers are retrieved and dispensed to said user through an opening.

2. The inhaler of claim 1, wherein the one or more retrieved liquids are at least partially admixed before being dispensed to said user.

3. The inhaler of claim 1, wherein the one or more retrieved liquids are dispensed separately to said user through the opening, wherein each of said plurality of chambers are directly connected to said opening at their one end.

4. The inhaler of claim 1, wherein liquid in a first chamber of said plurality of chambers is a medicament.

5. The inhaler of claim 4, wherein liquid in a second chamber of said plurality of chambers is an antidote of said medicament.

6. The inhaler of claim 5, wherein said inhaler comprises a communication module that is operatively coupled with a portable computing device, and wherein upon receipt of a second signal from said portable computing device, said antidote is mixed with said medicament.

7. The inhaler of claim 5, wherein said inhaler comprises a communication module that is operatively coupled with a portable computing device, and wherein upon receipt of a third signal from said portable computing device, said first chamber comprising the medicament is ruptured or rendered inaccessible to said user, said third signal being transmitted from the portable computing device when operation of said inhaler is not intended.

8. The inhaler of claim 1, wherein said liquid is in gaseous form.

9. The inhaler of claim 1, wherein said liquid is any or a combination of a herbal oil or a wax.

10. The inhaler of claim 1, wherein said liquid is cannabis oil or cannabis extract.

11. The inhaler of claim 1, wherein said inhaler comprises a communication module that is operatively coupled with a portable computing device, and wherein said inhaler is actuated upon receipt of a signal from said portable computing device.

12. The inhaler of claim 1, wherein said inhaler is configured in a manner such that the one or more retrieved liquids from the respective chambers are mixed in a configurable proportion, said configuration of liquid proportion being done through any or a combination of an interface provided on the inhaler, or through one or more signals sent by a portable computing device that said inhaler is operatively coupled to.

13. The inhaler of claim 1, wherein said container is made of any or a combination of a glass, plastic, or a metal.

14. The inhaler of claim 1, wherein said dosing parameter is selected by the user through any or a combination of an interface provided on the inhaler, or through one or more signals sent by a portable computing device that said inhaler is operatively coupled to, wherein said one or more signals are transmitted based on selection of an option through an application that is configured in the portable computing device.

15. The inhaler of claim 1, wherein said dosing parameter is pre-configured and automatically selected upon actuation of the inhaler.

16. The inhaler of claim 1, wherein said dosing parameter is indicative of any or a combination of liquids that need to be combined during dispensation through the opening, whether said liquids are to be dispensed as a combination or separately, and proportion/strength of each liquid to be dispensed.

17. The inhaler of claim 1, wherein said inhaler comprises a rotary device configured to rotate the container, and position the one or more chambers of the container for dispensing the selected liquids based on the received dosing parameter.

18. A method to dispense one or more liquids through an opening of an inhaler, said method comprising the steps of:
receiving, at the inhaler, an actuation signal to activate said inhaler, said inhaler comprising a container that further comprises a plurality of chambers, each chamber of said plurality of chambers being filled with a liquid; and
receiving, at the inhaler, selection of a dosing parameter based on which one or more liquids from respective chambers are retrieved and dispensed to said user through an opening

19. The method of claim 18, wherein the one or more retrieved liquids are at least partially admixed before being dispensed to said user.

20. The method of claim 18, wherein the one or more retrieved liquids are dispensed separately to said user through the opening, wherein each of said plurality of chambers are directly connected to said opening at their one end.
